(19) United States
(12) Patent Application Publication
(10) Pub. No.: US 2023/0149647 A1
(43) Pub. Date: May 18, 2023

(54) CUSTOMIZED FACE MASK MADE USING ADDITIVE MANUFACTURE

(71) Applicant: EOS of North America, Inc., Novi, MI (US)

(72) Inventors: R. Scott Evans, Novi, MI (US); Brian R. Golden, Novi, MI (US); Mohit Chaudhary, Novi, MI (US); Donald Vanelli, Novi, MI (US); Richard B. Denison, Novi, MI (US)

(73) Assignee: EOS of North America, Inc., Novi, MI (US)

(21) Appl. No.: 17/915,092

(22) PCT Filed: Mar. 31, 2021
(86) PCT No.: PCT/US21/25144

§ 371 Date: Sep. 27, 2022

Related U.S. Application Data
(60) Provisional application No. 63/003,806, filed on Apr. 1, 2020.

(51) Int. Cl. A61M 16/06; A61M 16/10; A61M 16/20; B29C 64/393; G06F 30/17

(52) U.S. Cl. CPC A61M 16/0605; A61M 16/1065; A61M 16/209; B29C 64/393; G06F 30/17; A61M 2016/0661; B33Y 80/00

(57) ABSTRACT

A face mask including a base model of a mask body having a front side and a back side and defining an interior. The back side includes a face opening within which a portion of a user face including a nose and mouth can be inserted and encompassed. The face opening is defined by a perimeter and is modified to substantially correspond to contours of the user face based on scan of topographical facial features of the user face.

### CROSS REFERENCES TO RELATED APPLICATIONS

This application claims priority to, and the benefit of, U.S. Provisional Patent Application Ser. No. 63/003,806, filed Mar. 31, 2020, the contents of which are incorporated herein by reference in their entirety.

### TECHNICAL FIELD OF THE INVENTION

The present invention relates generally to face masks. More particularly, the present invention relates to 3D printed face masks.

### BACKGROUND OF THE INVENTION

Face masks, such as surgical face masks, are often worn by health care professionals to protect themselves and patients. Face masks can capture bacterial and viral particles dispelled from the wearer's mouth and nose during exhalation. The human face presents a challenge for forming a seal between a face mask and the face. The human face is deeply contoured, and the size and proportion of these contours vary widely between human faces. However, face masks are generally loose fitting, thereby allowing bacterial and viral particles present in exhalation gases to flow around the perimeter of the face mask, such as at the lower edges of the cheeks and around the chin of the wearer.

Conventional 3D printed face masks also fail to adequately seal to the face of a wearer due to the more rigid nature of conventional 3D printed materials. Moreover, softer 3D printed materials that are available are not easy to stretch, which risks seal failure when the user is talking, moving, temperature changes, or even body moisture changes.

### SUMMARY OF THE INVENTION

The present invention relates broadly to a 3D printed mask for respirator use that is customized to a face of a user to ensure high quality sealing using a method. The mask may further be made from materials that allow cleaning and sterilization, for reusability. The method captures face scans from portable computing devices, such as, for example, mobile phones, tables, etc., having cameras. The face scans are converted into facial models. The facial models are assessed for facial features that allow for creating a custom contoured facial mask from a standard facial mask set of patterns. The patterns are selected and scaled to the face of the user. The sealing contour of the mask is aligned and matched to the contour of the user's face to ensure a good seal. Alternately, the facial scan is used to create a generative facial mask design starting from the facial scan and building outward to a full custom facial mask model. The present invention results in better sealing and improved comfort for the wearer compared to current designs.

In an embodiment, the present invention broadly includes a method of making a custom contoured facial mask. The method includes scanning a face of a wearer for facial scan data, evaluating the facial scan data, selecting a standard facial mask design from a library of patterns, scaling the selected pattern to a size of the face, aligning the selected pattern to the facial scan, merging the selected pattern onto the face, and creating a contour on a sealing edge of the selected pattern that corresponds to the face.

In an embodiment, the method further includes conducting modifications to the final custom contoured facial mask, such as, for example, providing different contour profiles, moving or otherwise relocating anchor points on the facial mask, adjusting various feature inserts, such as, for example, one way exhalation valves to relieve pressure on the user while breathing in a sealed mask, adjusting filter cartridge designs or other features as may be desired. The method can also further include exporting a 3D printable file that is usable (printable) by a 3D printer, enabling the printing in a 3D printer by a set of build instructions for the printer, such as, for example, scanning strategies, part placement, and printing the 3D printable file in a 3D printer.

In another embodiment, the present invention broadly comprises a computer implemented method for managing data collection, data conversion, data privacy and data deployment and matching scans with available printer capacity to rapidly and efficiently enable distributed production of face masks.

In another embodiment, the present invention broadly comprises a face mask including a generally cup or ovoid shaped mask body having one or more airflow openings. One side of the face mask is open and shaped to receive the mouth and nose features of a human face. The side has a peripheral edge shaped to generally follow a contour of a human face extending around the mouth and nose areas. The shape of the peripheral edge is most advantageously customized to a specific facial features of the user. However, the face mask could also be made in a variety of standardized facial features/contours, which can then be selected for best-fit.

While it is considered that a face mask according to an aspect of the present invention may not need any kind of separate gasket-like element for sealability to the face of the user, in an embodiment a soft or otherwise pliant seal is coupled, for example, to a peripheral edge. The seal may extend around the entirety of the peripheral edge, or for at least a portion thereof to enable necessary comfort and seal.

In an embodiment, the airflow opening includes filter material. One or more valves may be incorporated, such as, for example, at least one exhalation valve that is disposed on the mask body and has at least one orifice that allows exhaled air to pass through the mask body to ambient air. In an embodiment, at least one inhalation valve is disposed on the mask body and has at least one orifice that allows inhaled air to pass through the mask body for the user to breathe.

### BRIEF DESCRIPTION OF THE DRAWINGS

For the purpose of facilitating an understanding of the subject matter sought to be protected, there are illustrated in the accompanying drawings embodiments thereof, from an inspection of which, when considered in connection with the following description, the subject matter sought to be protected, its construction and operation, and many of its advantages should be readily understood and appreciated.

FIG. 1 is an exploded perspective view of an exemplar face mask incorporating an embodiment of the present invention.

FIG. 2 is an assembled perspective view of an exemplar face mask incorporating an embodiment of the present invention.

FIG. 3 is a perspective view of an exemplar face mask incorporating another embodiment of the present invention.

FIG. 4 is a detailed perspective view of an exemplar face mask having user-identifier indicia incorporating another embodiment of the present invention.

FIG. 5 illustrates multiple face masks made in a single build of an additive manufacturing (AM) machine.

FIG. 6 is a detailed, perspective view of the face mask illustrated in FIG. 3.

FIG. 7 is another perspective view of the face mask illustrated in FIG. 3.

FIG. 8 is another perspective view of the face mask illustrated in FIG. 3.

FIG. 9 a perspective view of an exemplar filter compartment cap incorporating another embodiment of the present invention.

FIG. 10 is a flow chart illustrating an exemplary method of 3D printing of face masks incorporating an embodiment of the present invention.

FIG. 11 is a flow chart illustrating an exemplary method of customizing a face mask incorporating an embodiment of the present invention.

FIG. 12 is a schematic illustration of computer hardware functionality that implements aspects of at least some of the presently disclosed embodiments.

### DETAILED DESCRIPTION OF THE INVENTION

While the present invention is susceptible of embodiments in many different forms, there is shown in the drawings, and will herein be described in detail, embodiments of the invention, including a preferred embodiment, with the understanding that the present disclosure is to be considered as an exemplification of the principles of the present invention and is not intended to limit the broad aspect of the invention to any one or more embodiments illustrated herein. As used herein, the term “present invention” is not intended to limit the scope of the claimed invention, but is instead used to discuss exemplary embodiments of the invention for explanatory purposes only.

The present invention relates broadly to face masks, such as, for example, face masks that can be used in a surgical field. However, the present invention is not limited to such use. The face mask includes a custom fitting reusable mask body, one or more filtered openings, which may further include a valve(s), and an adjustable detachable strap system. The face mask invention is most preferably manufactured using Additive Manufacture (AM) techniques, such as, for example, selective laser melting (SLS), Fused deposition modeling (FDM), stereolithography (SLA), etc. Another embodiment of the present invention is a method of capturing and using facial data to customize a face mask.

Additive Manufacturing (AM) is well known as a general subject. Reference can be made to U.S. Pat. No. 10,259,041, for example, the contents and teachings of which are incorporated herein in their entirety. With regard to the AM process and system of powder bed fusion, this involves a manufacturing method for generatively manufacturing of a three-dimensional (3D) object by layer-by-layer application and selective solidification of a building material, preferably a powder, including the steps of: applying a layer of the building material to a build area by means of a recoater, selectively solidifying the applied layer of the building material at points corresponding to a cross-section of the object to be manufactured by means of a solidification device, and repeating the steps of applying and solidifying until the three-dimensional object is completed. As noted throughout this specification, however, the present invention has application beyond just the powder bed fusion process, and can be implemented using all types of similar layerwise manufacturing techniques. Moreover, it will be understood that application of aspects of the invention can be practiced outside of the 3D printing.

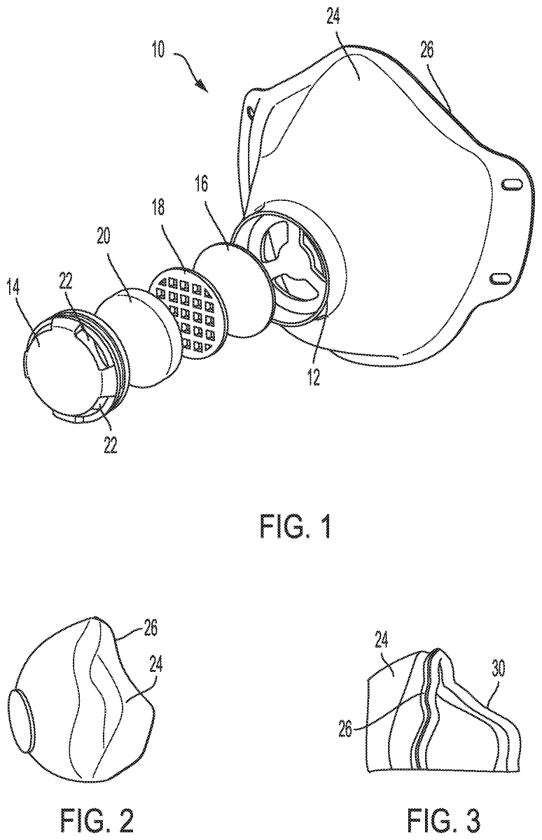

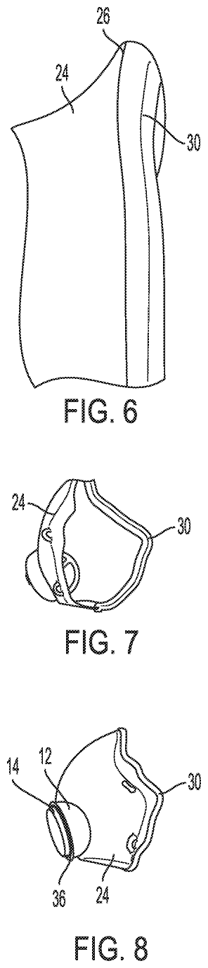

Referring to FIGS. 1-8, an exemplary face mask 10 incorporating an embodiment of the present invention is depicted. The face mask includes a filter compartment 12 adapted to receive standard-type filter elements. A cap 14 is adapted to close the filter compartment 12 to retain the filter elements. Filter elements can include a fabric layer 16, foraminous filter holder 18, main filter media 20, as well known in the art. Cap 14 includes cap openings 22 adapted to allow air to pass through the face mask 10. In an embodiment, the filter elements include a filter that can qualify as an N95 filter, a N99 filter, or any other NIOSH rating, such as, for example, P100 and OV-100.

The face mask 10 includes a body 24 that has been extruded based on a facial scan to create a contour 26 that matches a user face, as described below. The body 24 includes the filter compartment 12, cap 14, and filter elements. The contour 26 can be further profiled to create different edge features. For example, the contour 26 could be thinned to create a knife-like edge adapted to be inserted into a seal 30 to provide further tolerance and cushioning at the areas of contact between the face mask 10 and the face of a user. Referring to FIGS. 3 and 6-8, an exemplary contour 26 inserted into a non-3D printed seal 30 is illustrated. The seal 30 is composed of a material softer than the body 24, such as, for example, a rubber or silicone seal 30.

In an embodiment, the seal 30 is composed of an elastomeric material, such as, for example, a thermoplastic elastomer (TPE), a foam composed of, for example, polyurethane, a combination of materials, such as, for example, TPE with ethylene-vinyl acetate (EVA) and/or polyurethane (PU), or any material suitable for forming an air-tight seal between the face of the user and the face mask 10. In another embodiment, the foam of the outer seal comprises polyurethane. Different contours 26 can be designed into the 3D printed face mask 10 to accept different types of seals 30. In another embodiment, the contour 26 is designed such that no intermediate material (i.e., seal 30) is necessary. In an embodiment, the seal 30 is disposed on only a part of the contour 26, such as at an are proximate the bridge of the nose of the user, and then tapered to meet the cheek of the user below the nose area. The design of the contour 26 and use of the seal 30 is a matter of facial geometry and comfort.

In an embodiment the contour 26 is rounded in the shape of a teardrop, dumb-bell or other suitable shape that interfaces directly to the face and creates a softer impact on the skin while allowing for some movement of the face mask 10 (i.e., rolling, sliding) while still maintaining a seal. The contour 26 could have an “S shape” that if managed with the AM printing process and suitable polymer build material, such as, for example, PA 2200, which is a standard polymer material in AM processes. The build material may have approximately 0.4 mm wall thicknesses and appropriate curvature to creates a biasing effect at the interface with the face, thereby allowing the mask to move and flex to maintain a seal as the wearer moves his/her face or if the facial contour changes slightly due to blood pressure, skin moisture, temperature, etc. This also further reduces the pressure by which the mask must be pulled onto the face with attachment mechanism, such as, for example, straps. Due to variability in facial features, a need for different basics shapes may be needed to fit certain populations. A basic design could vary in areas such as the flair over the nose area to develop space between the nose and the wearer depending on facial features to enable comfort and seal of the mask. In another embodiment, the contour 26 could be a 3D lattice structure that also provides the aforementioned biasing effect at the interface of the mask contour 26 with the face. Such lattice structures made from resilient polymer material manufactured by AM, such as, the Digital Foam line by EOS GmbH Electro Optical Systems.

The face mask 10 can be made from many different materials. Preferably, a material is chosen to allow for a re-usable mask shell, therefore the material should have certain qualities that allow it to be cleaned and/or sterilized with some periodicity. For example, PA 2200 is a prime candidate for this application. PA 2200 is a nylon-12 material that is well known in the additive industry for its qualities of high lot to lot consistency from the supplier and a good balance of physical strength, surface detail, and ease of processing in the 3D printers. Furthermore, PA 2200 is currently used in various medical device applications with associated FDA approvals, including surgical cutting guides, where it is rated for exposure for various pathogens, is sterilizable, is capable of managing temperatures of 100 degrees C. to 140 degrees C. without significant degradation (in a typical autoclave temperature range), and can be cleaned with soap and water. Additionally, PA 2200 is easy to manage curves and other generally challenging physical profiles that can be generated in 3D printing. Thin, curved surfaces are particularly advantageous in that they give the mask some ability to flex when bent and at the same time are easy to clean for wearers as compared to sharp angles and corners.

In an embodiment (best illustrated in FIG. 8), a slight ridge or bead 36 is formed on the filter cap 14 or the filter compartment 12 to allow for alternate filter materials to be used. The bead 36 forms a shoulder to capture a tie-down element for attaching a filter element over the filter compartment 12. For example, the 36 bead allows the use of either a filter under the cap 14 and screwed into the mask, or the ability to dispose a filter material over the compartment 12 opening and secure it with a tie underneath the bead 36. This is especially useful in times of shortages of filter material when alternatives are needed. For example, an existing N95 fabric mask could be cut up and a piece placed over the cap opening and then secured by a strap around under the ridge or bead 36.

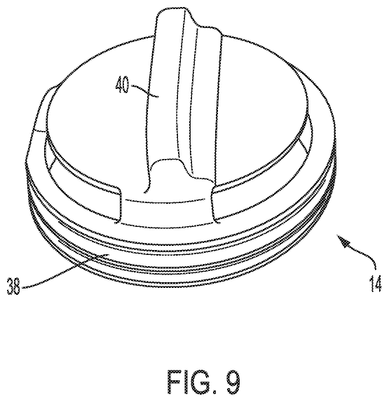

Referring to FIG. 9, the filter cap 14 includes a raised part or bar 40 formed on the outside of the cap 14 for manipulation by the user to enable the filter cap 14 to be removed while reducing the amount of touch contact a user would have to have with the rest of the surface of the face mask 10. This feature is especially useful in infectious disease environments where virus particles are present on surfaces. The raised part 40 thus enables easy removal and insertion. The cap 14 includes a threaded portion 38 adapted to treadably couple with corresponding threads within the filter compartment 12. The cap 14 can be made simultaneously with the body 24 of the face mask 10 in the AM process.

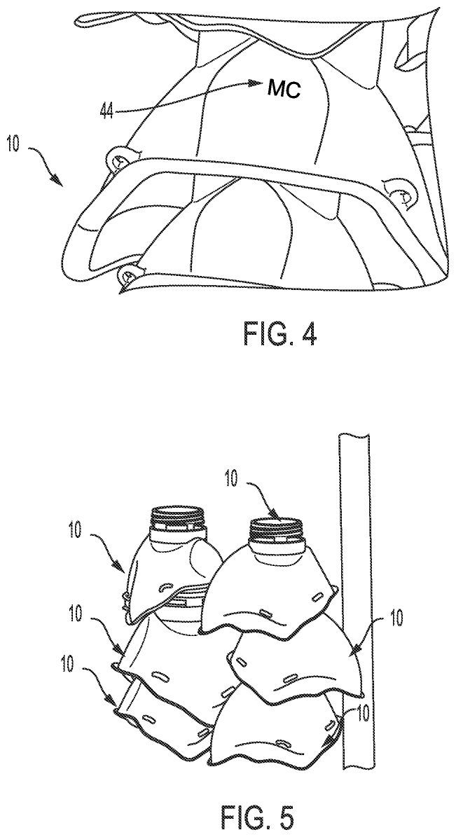

In another embodiment as illustrated in FIG. 4, the face mask 10 includes a unique identifier 44 printed on the face mask 10. For example a user's (for example, a doctor, nurse, clinician, or the like) initials, name, and/or a mask identification number that could then allow cleaning, reuse, and tracking in a clinical environment, thereby reducing risk of using someone else's mask, or even allowing different mask numbers to be used only in certain patient environments, for instance used only for Patient A's room.

In another embodiment, the face mask 10 includes an exhalation valve, such as, for example, a one-way valve. The exhalation valve allows exhaled gases to bypass the filter due to less flow resistance, thereby providing greater comfort and less vapor-induced fogging. Examples of such devices include U.S. Pat. Nos. 7,188,622, 4,873,972, and 5,509,436, for instance. Preferably, a design of a one-way valve would be applicable to an operating room environment, where the exhaled products are filtered.

3D printers, such as those built and sold by EOS GmbH Electro Optical Systems, as selective laser sintering (solidifying) printers, have a certain amount of build volume available in which to place parts for printing. The printers also must have instructions on how to build the desired parts, such as how thick or thin should each layer be, what laser power must be used, how must the laser scan the part (e.g., in what direction, what number of passes, what patterns, etc.), what temperatures, recoating techniques, and various other common settings are required to ensure a successful part as a result. The operation of such printers is well known in the art, and detailed description thereof is unnecessary herein.

The arrangement of the parts in the build, such as, placement, orientation, location next to each other, etc., can affect the final part quality with regards to feature definition, properties of the part (mechanically and other like surface quality) and dimensions, as well as how fast or how many parts can be placed in each run of the 3D printer.

A build instruction kit is therefore another aspect of the invention that is created for the 3D printer to optimally print the face mask 10. This would include a build setup, which can be automated, such a process including selecting the desired parts to be printed, importing the parts into a 3D printer CAD software environment, such as, for example, Magics by Materialise, and then allowing the software to automatically place and align the parts in an optimal fashion subject to rules and conditions set by the 3D printer operator or process developer. FIG. 5 illustrates a possible result of such a setup. Doing an automated process both reduces the time required by a 3D printer operator to prepare a machine for printing parts, thereby increasing productivity, and increases the quality of the final parts.

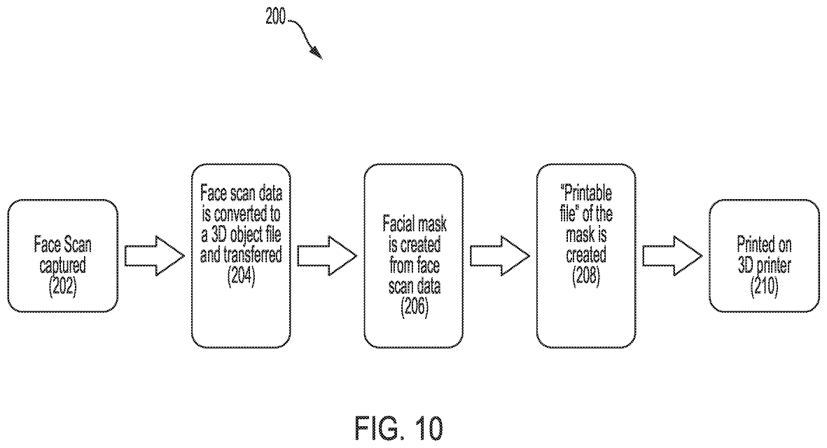

Referring to FIG. 10, a flow chart of a method 200 for managing the data from face scan to 3D print is illustrated. Face scan data is captured using a computing device, such as a phone, tablet, computer, etc., via a software application, such as, for example, bellus3D (www.bellus3d.com), illustrated at step 202. The face scan data including topographical facial features of a person The face scan data is converted to a 3D object file and transferred, such as, e.g., via e-mail, to a data exchange, illustrated at step 204.

The 3D object file is stored and/or converted into a printable face mask file. The face scan data can include 3D model data such as a point cloud. Example file formats include, but are not limited to, STL, OBJ, FBX, COLLADA, 3DS, IGES, STEP, and VRML/X3D. Several elements of additional data (e.g., metadata) would also need to be transferred with the 3D object file. For example, user information (e.g., name, desired mask label, etc.), purchaser information, privacy acknowledgements and rights for the exchange to store the information or associated limitations on its use, desired shipping information and timing, etc.

A facial mask design is created from the facial scan data, illustrated at step 206, including topographical facial features of a person. Additional calculations could be made from the facial scan, such as, for example, comparing the face to a reference set of faces to understand symmetry and sizing that may affect the final customized facial mask selection and design. The facial mask design can be made using a computer aided design (CAD) application. CAD applications are well known in the art and are not described in detail herein.

Finally a printable file would then be made available on the data exchange based on the facial mask design, illustrated at step 208. The printable file could be made available to the purchaser, the user whose face was scanned, and/or published on a marketplace for an owner of 3D printer to select and fulfill the order (print, verify quality, deliver) via a computing device electronically communicable with the data exchange using known methods, illustrated at step 210. Various order status information could also be managed on this data exchange platform. Although the present embodiment is described in relation to face masks, the invention is not limited as such and could also be used to 3D print an array of other personalized products, such as, for example, gloves, glasses, helmets, braces, wearables, etc.

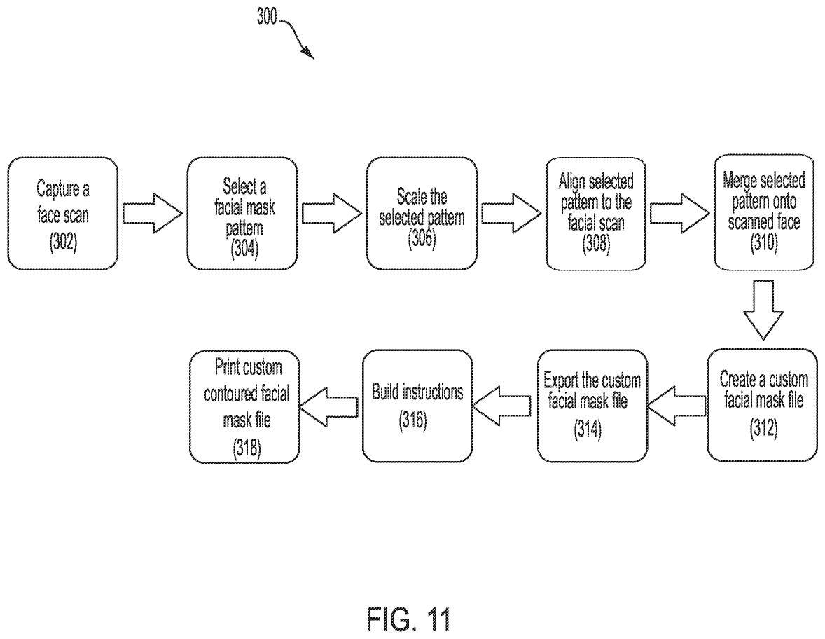

Referring to FIG. 11, an exemplary method 300 of customizing a face mask incorporating an embodiment of the present invention is illustrated. One aspect of the method 300 is the capture and then use of facial data to customize the face mask 10, or respirator. The method 300 includes capturing topographical facial features of a person in a face scan using a computing device, such as a phone, tablet, computer, etc., via a software application, such as, for example, bellus3D (www.bellus3d.com), illustrated at step 302. Selecting a facial mask pattern from a database, illustrated at step 304. Scaling the selected pattern to the size of the scanned face, illustrated at step 306. The step of scaling the selected pattern may be accomplished by the user manually selecting topographical facial features using an user interface in a well-known manner. The selected pattern is aligned to the facial scan in a CAD environment, illustrated at step 308. The selected topographical facial features are used to align the selected pattern to the facial scan. The selected pattern is then merged onto the scanned face, illustrated at step 310. A custom facial mask file having a contour on the sealing edge of the mask is created that substantially corresponds to the topographical facial features of the scanned face, illustrated at step 312. The custom facial mask file may be modified by, for example, providing different contour profiles, moving or relocating the selected topographical facial features, adjusting various feature inserts, such as, for example one way exhalation valves, adjusting filter cartridge designs or any other features as may be desired. The custom facial mask file is exported to a 3D printable custom contoured facial mask file that is usable (printable) in a 3D printer, illustrated at step 314. The printing in a 3D printer is enabled by a set of build instructions for the printer (e.g., scanning strategies, part placement) in a well-known manner, illustrated at step 316. The 3D printable custom contoured facial mask file is then printed by a 3D printer using an AM technique according to the build instructions, illustrated at step 318.

In similar fashion, the same basic method as the custom contoured facial mask as described above can be used for what is referred to as a generative type face mask. For this adaptation, the facial scan is used to create a full generative design. Thus, instead of selecting a facial mask pattern from a database, illustrated at step 304, this method uses the facial scan to provide the starting surface (plane) from which software algorithms then grow a facial mask off of the face, thereby creating a fully custom facial mask design that is unique to the individual's face.

Such a generative facial mask design could incorporate “standard elements” as well, such as, a filter cartridge holder for which a standard size would be desirable, in so much as this would allow ready mass and efficient production of re-usable elements like filter material, such as, a consumable cartridge that is changed out between a certain number of uses. While generative design is known, current art does not customize or personalize the final design to facial features of an individual. Rather, typical generative design is used for parts manufacturing, custom product design, and even floorplan design.

By enabling a digital build model and set of instructions for 3D printers, it is possible to rapidly deploy and scale up a distributed production model. For example, once the face mask print files are created and a set of instructions to run the printers created, such data can be digitally made available (via the internet, etc.) to physical sites where 3D printers are located. In this way, new models and designs can be rapidly deployed to points of production. This is especially useful in scenarios, such as natural disasters or epidemics, where supply chains and traditional logistics may be highly disrupted. For example, if digital build information can be deployed to a region with 3D printers, then items that cannot be obtained otherwise could be built locally, bypassing traditional supply chain hurdles. Additive manufacture (AM) of this type also enables the ability to modify designs extremely rapidly, essentially in real time, to adapt to evolving needs.

For example, in the event of an infectious disease outbreak, a digital build kit can be deployed to sites that are located in the outbreak concentration areas with 3D printers and appropriate raw material stocks. The stocks can be converted to the items of most need.

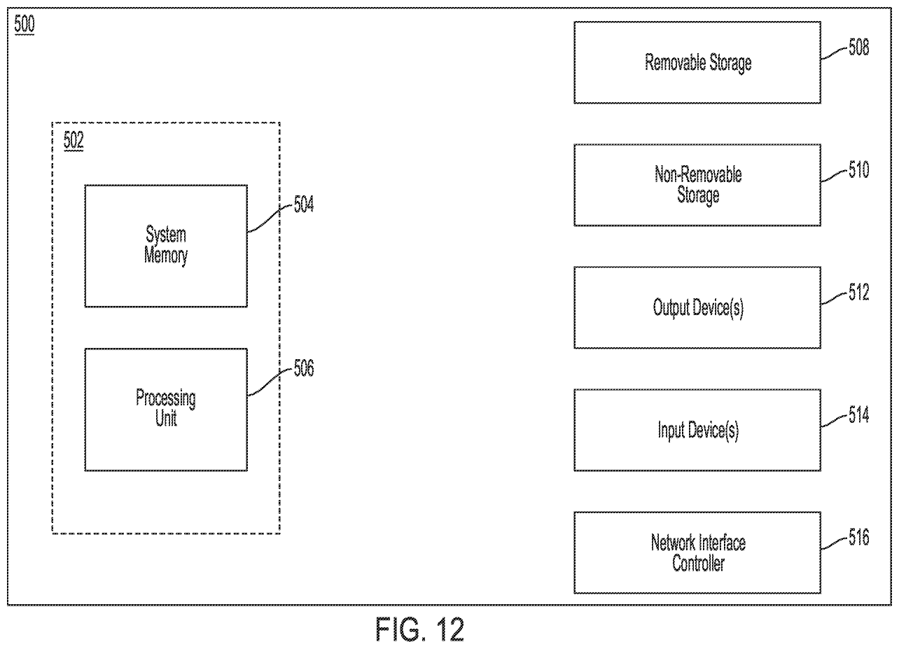

Referring to FIG. 12, an example computing device 500 upon which embodiments of the invention may be implemented is illustrated. It should be understood that the example computing device 500 is only one example of a suitable computing environment upon which embodiments of the invention may be implemented. Optionally, the computing device 500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 500 typically includes at least one processing unit 506 and system memory 504. Depending on the exact configuration and type of computing device, system memory 504 may be volatile (such as random access memory (RAM)), non-volatile (such as read-only memory (ROM), flash memory, etc.), or some combination of the two. This most basic configuration is illustrated in figure below by dashed line 502. The processing unit 506 may be a standard programmable processor that performs arithmetic and logic operations necessary for operation of the computing device 500. The computing device 500 may also include a bus or other communication mechanism for communicating information among various components of the computing device 500.

Computing device 500 may have additional features/functionality. For example, computing device 500 may include additional storage such as removable storage 508 and non-removable storage 510 including, but not limited to, magnetic or optical disks or tapes. Computing device 500 may also contain network connection(s) 516 that allow the device to communicate with other devices. Computing device 500 may also have user input device(s) 514 such as a keyboard, mouse, touch screen, etc. Output device(s) 512 such as a display, speakers, printer, etc. may also be included. The additional devices may be connected to the bus in order to facilitate communication of data among the components of the computing device 500. All these devices are well known in the art and need not be discussed at length here.

The processing unit 506 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 506 for execution. Example tangible, computer-readable media may include, but is not limited to, volatile media, non-volatile media, removable media and non-removable media implemented in any method or technology for storage of information such as computer readable instructions, data structures, program modules or other data. System memory 504, removable storage 508, and non-removable storage 510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 506 may execute program code stored in the system memory 504. For example, the bus may carry data to the system memory 504, from which the processing unit 506 receives and executes instructions. The data received by the system memory 504 may optionally be stored on the removable storage 508 or the non-removable storage 510 before or after execution by the processing unit 506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of the inventors' contribution. The actual scope of the protection sought is intended to be defined in the following claims when viewed in their proper perspective based on the prior art.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It should be appreciated that the logical operations described herein with respect to the various figures may be implemented (1) as a sequence of computer implemented acts or program modules (i.e., software) running on a computing device (e.g., the computing device described in the figure below), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

What is claimed is:

1. A method for making a face mask comprising the steps of:
capturing topographical facial features of a face in a face scan;
selecting a facial mask pattern from a database;
scaling the selected facial mask pattern to a size of the face;
aligning the selected facial mask pattern to the face;
merging the selected facial mask pattern to the face;
creating a custom facial mask file of a face mask having a contour on a sealing edge of the face mask that corresponds to the topographical facial features of the face; and
exporting the custom facial mask file to a 3D printable custom contoured facial mask file that is usable in a 3D printer.

2. The method of claim 1 further comprising modifying the custom facial mask file.

3. The method of claim 1, further comprising inputting the 3D printable custom contoured facial mask file into an operating system of an Additive Manufacturing system, and operating the system to generatively build the face mask.

4. The method of claim 1, wherein the AM system is a powder bed fusion 3D printing process.

5. The method of claim 1, further comprising providing a seal adapted to receive a portion of the contour.

6. A face mask made in accordance with the method of claim 1.

7. A face mask made in accordance with the method of claim 2.

8. A face mask comprising:
a body defining an interior and having a contour that substantially corresponds to topographical features of a face captured by a face scan.

9. The face mask of claim 8, further including:
a filter compartment formed in the mask body; and
a cap removably coupled to the body and having cap openings adapted to allow air to pass through the face mask.

10. The face mask of claim 9, wherein the cap is threadably coupled to the body.

11. The face mask of claim 10, further comprising a bead formed around one of the cap and well, wherein the bead forms a shoulder to capture a tie-down element for attaching a filter element over the filter compartment.

12. The face mask of claim 8, further comprising a seal adapted to receive a portion of the contour

13. The face mask of claim 8, wherein the face mask is formed by an Additive Manufacturing system.

14. A computer-implemented method for making a face mask comprising the steps of:
receiving topographical facial features of a face captured in a face scan;
creating a custom facial mask file of a face mask having a contour on a sealing edge of the face mask that corresponds to the topographical facial features of the face; and
exporting the custom facial mask file to a 3D printable custom contoured facial mask file that is usable in a 3D printer.
